(19) United States
(12) Patent Application Publication
(10) Pub. No.: US 2004/0137608 A1
(43) Pub. Date: Jul. 15, 2004

(54) Chemical microarrays and method for constructing same

(76) Inventors: Aaron Garzon, Rehovot (IL); Rick Johnston, Murphys, CA (US); Rami Skaliter, Nes Ziona (IL)

Correspondence Address: John P. White, Cooper & Dunham LLP, 1185 Avenue of the Americas, New York, NY, 10036, US

(21) Appl. No.: 10/723,076

(22) Filed: Nov. 26, 2003

Related U.S. Application Data
(60) Provisional application No. 60/429,496, filed on Nov. 27, 2002.
(60) Provisional application No. 60/449,748, filed on Feb. 25, 2003.

(51) Int. Cl. G01N 33/53; C12M 1/34; G01N 33/543

(52) U.S. Cl. 435/287.2; 435/7.1; 436/518

(57) ABSTRACT

The present invention relates to a novel microarray, which may be used to present chemical molecules, and methods of preparing said microarray and screening said microarray.

The microarrays of the present invention may be used in various applications, such as, inter alia, drug discovery.

### BACKGROUND OF THE INVENTION

Screening for small molecules based on binding is an important aspect of modern biology and biochemistry. The possibility of identifying, for example, small molecule inhibitors of proteins can lead to advances in the field of drug discovery. One possibility of screening for small molecules involves using a small molecule array (SMA) which is a relatively new technology.

An array is an orderly arrangement of samples. It provides a medium for matching known and unknown samples based on biochemical interactions and automating the process of identifying the unknowns. An array experiment can make use of common assay systems such as microplates or standard blotting membranes, and can be created by hand or by use of robotics to deposit the sample. Microarrays available include oligonucleotide/DNA microarrays (see U.S. Pat. Nos. 6,291,170; 5,807,522 (inventors Shalon and Brown); U.S. Pat. Nos. 6,110,426; 5,716,785 and 5,891,636, (inventors Eberwine et al) all assigned to Board of Trustees of Leland Stanford University and U.S. Pat. No. 4,981,783 (inventor Augenlicht), polypeptide/protein microarrays (see Ekins R. P., J Pharm Biomed Anal 1989. 7: 155; Ekins R. P. and Chu F. W., Clin Chem 1991. 37: 1955; Ekins R. P. and Chu F. W, Trends in Biotechnology, 1999, 17, 217-218; G. MacBeath and S. L. Schreiber, Printing Proteins as Microarrays for High-Throughput Function Determination, Science 2000 Sep. 8: 289(5485): p. 1760-1763) and, as will be described below, chemical/small molecule microarrays.

Chemical Microarrays

Small Molecule Arrays, or chemical microarrays or SMA, are microarrays with low molecular weight compounds bound on a planar format at high spatial frequency. SMAs containing thousands or even tens of thousands of unique chemical compounds attached to a biologically inert supporting surface, offer great promise for the facile identification of agents that modulate either protein activity or gene transcription. Proteins generally have binding sites for small molecules for important biological reasons. These molecules may act as substrates, inhibitors, activators, or even transcriptional regulators of the protein, interacting through one or more independent binding sites on the polypeptide. Upon binding in-vivo, such small molecules (and possibly specific chemical analogs thereof) exert their influence on specific biochemical pathways.

SMAs are screened (e.g. for a polypeptide probe) essentially according to the following method: the chemical compounds are “probed” with one or more protein molecules, and a detection methodology reveals those regions of the array (or spots) that are enriched in the selected protein(s). Spots that are enriched in the concentration of a particular probe represent a binding event of the selected protein to the underlying attached small molecule (or element). In this manner, high-density SMAs offer the possibility of probing thousands of small molecules for their ability to be recognized by a selected protein in a single experiment.

SMAs are predicted to have great utility in helping to identify promising new drug candidates or “hit molecules” when used in conjunction with specific protein(s) known to be involved in a particular disease state or metabolic process. When combined with directed medicinal chemistry efforts, a SMA could also help refine the structures of existing pharmaceuticals to make them safer and/or more effective. In addition, a SMA could also be potentially employed to identify specific protein(s) through which a pharmacologically-active compound or mixture exerts its influence. Once identified (via sensitive micro-sequencing or mass spectral techniques of the protein recovered from the array), the protein could be produced in larger quantities by molecular biology techniques and then subsequently be used to search for better and more effective modulators of its activity in the manner briefly described above.

There are in general two ways one could manufacture a SMA, with various permutations yielding a wide variety of approaches. The first is to deposit or “print” already synthesized molecules to the surface where they are immobilized in some fashion prior to being probed. The other approach employs an in situ synthesis regime where the small molecules are directly synthesized on the surface using photolithographic techniques (Fodor, S P, Read, J L, Pirrung, M C, Stryer, L, Lu, A T, and Solas, D. (1991). Light-directed, spatially addressable parallel chemical synthesis. Science. 251:767-773) or so-called “mask-less” technologies (Singh-Gasson, B, Green, R D, Yue, Y, Nelson, C, Blattner, F, Sussman, M R, and Cerrina, F. (1999). Maskless fabrication of light-directed oligonucleotide microarrays using a digital micromirror array. Nat. Biotechnol. 17:974-978.; LeProust, E, Pellois, J P, Yu, P, Zhang, H, Gao, X, Srivannavit, O, Gulari, E, and Zhou, X. (2000). J. Comb. Chem. 2:349-354.). Both methods have been successfully used in the preparation of several kinds of microarrays.

Several publications demonstrate the use of small molecule microarrays: MacBeath et al (MacBeath G, Koehler, A N, and Schreiber S L (1999). Printing small molecules as microarrays and detecting protein-ligand interactions en masse. J. Am. Chem. Soc. 121:7967-7968.) prepared a series of test compounds with thiol-containing “tethers,” and using precision robotics, printed them in DMF directly to a glass slide that was derivitized to contain active maleimido groups on the surface. Post deposition, the sulfhydryl group reacted with the maleimido group to form the respective thioethers immobilizing the compounds in 200-250 um diameter spots. The authors then used 2-mercaptoethanol to react with the excess maleimide in order to prevent non-specific target protein binding to the surface mediated by any cysteines present within the protein. The spotted glass slides were subsequently probed with a series of fluorescently-tagged cognate proteins and scanned for fluorescence. The authors demonstrated not only specificity in the recognition of protein-ligand binding for three different model systems, but also that signal generation over any spot was entirely dependent on their specifically designed surface “capture chemistry.”

Hergenrother et al (Hergenrother, P J, Depew, K M, and Schreiber, S L (2000). Small molecule microarrays: Covalent attachment and screening of alcohol-containing small molecules on glass slides. J. Am. Chem. Soc. 122:7849-7850.) showed that compounds containing a primary alcohol function could be covalently attached after printing onto glass surfaces derivatized with thionyl chloride, and detection of cognate protein binding was demonstrated using the same basic model systems and fluorescence scanning techniques employed by MacBeath et al (see above). Hergenrother et al illustrate that SMA manufacture can be successfully interfaced with the results of split-pool combinatorial chemistry in screening the large number of compounds potentially available.

Kuruvilla et al (Kuruvilla, F G, Shamji, A F, Sternson, S M, Hergenrother, P J and Schreiber, S L. (2002). Dissecting glucose-signaling with diversity-oriented synthesis and small molecule microarrays. Nature 416:653-657.) illustrated that a SMA could be used to simultaneously screen thousands of molecules derived from an “on-bead” combinatorial synthetic effort for their ability to be recognized and bound to a Cy5-conjugated yeast protein called Ure2p. The authors generated their diversity-oriented compound libraries in microtiter plates on individual macrobeads, then released the compounds from the beads prior to robotically printing 1 ul of each in DMF at a final density of 800 spots/square cm on a thionyl chloride-derivitized glass surface. One of the eight recognized compounds, when subsequently added back to live yeast cultures, activated a glucose-sensitive transcriptional pathway downstream of Ure2p (but known to be modulated by it). These results represent the first demonstration of new “hits” being generated against a specific protein using SMA technology.

In MacBeath et al (see above), the capture chemistry consisted of synthetically introducing a sulfhydryl-containing tether to their model compounds and reacting the products with maleimido-functionalized glass. The activated glass had to be prepared by the authors in a multistep process: cleaning, reaction with 3-aminopropyltriethioxysilane, and further reaction with the bifunctional reagent N-succinimidyl 3-maleimido propionate.

In the more general sense, any molecule without the specific functional group required for reaction with the selected capture chemistry on the surface will likely diffuse away and yield no signal, even if the molecule had the potential of being recognized by the target protein. This would lead to false negatives. In addition, the very process of attachment could inactivate subsequent protein binding if the unmodified form of the functional group was required for recognition by the protein. In this case, another false negative would be generated. Hence, surfaces that have two or more capture chemistries capable of operating simultaneously and without interference from each other would be very useful.

More likely is the case where many molecules within a given library will have a multiplicity of different functional groups such that capture chemistries can be potentially designed to attach the molecule to a surface in a variety of different ways. Attachment of otherwise identical molecules to surfaces through different functional groups alters the subsequent “presentation” of that molecule to the polypeptide probe, and is highly desirable as a means of avoiding false negatives as described above.

PCT patent application publication numbers WO 99/13313 and WO 02/05945 disclose different methods for producing microarray chips imprinted with a variety of test substrates; in addition, WO 00/71746 discloses a method of producing a microarray of chemical compounds.

### SUMMARY OF THE INVENTION

The present invention discloses a novel microarray, capable of presenting almost any existing chemical library (containing chemical molecules with varying functionalities), and methods of preparing said microarray. In a preferred embodiment of the present invention, this novel microarray is used in order to screen for chemical molecules that bind a polypeptide probe.

### DETAILED DESCRIPTION OF THE INVENTION

One embodiment of the present invention is directed to a multi-functional microarray comprising the following elements: a solid support; a series of wells contained on the solid support; a plurality of linkers attached to each well; a first functionality bound to a portion of the linkers within the plurality of linkers; and a second functionality bound to a different portion of the linkers within the plurality of linkers. The functionalities may be spatially separated; optionally, a portion of the plurality of linkers have no bound functionality, thereby achieving spatial separation between the functionalities, i.e., the spatial separation is possibly achieved by interspersing “no functionality” linkers with the “functionality” linkers.

By “linker” is meant any molecule of variable length, that is capable of binding a solid support and an additional entity, primarily a functionality, so that the solid support and the functionality are, essentially, bound together indirectly. The term linker also includes molecules which are capable of binding a solid support and possess an inherent functionality (the functionality being capable of reacting with a chemical entity—see below).

By “functionality”, in the context of the instant application, is meant a chemically reactive group comprising a small molecule capable of binding a linker and subsequently reacting with a chemical entity, and/or capable of reacting with a chemical entity and subsequently binding a linker. The reaction between the functionality and the entity leads to the formation of a bond, optionally a covalent bond. In one embodiment, the functionality is not a nucleic acid or a polypeptide or an antibody.

It will be understood that, in the context of the present invention, the functionality may also be an integral part of the linker molecule (according to the selection of specific linkers—see Example 1), and is not necessarily a separate chemically reactive group that must be chemically attached to the linker, but, rather, a chemically reactive group contained in the linker.

The term “capture chemistry” describes the process by which a functionality reacts with a chemical entity.

“Spatial separation” in the context of the present invention means that the linkers/functionalities are sufficiently separated from each other so that a chemical entity being bound to a functionality is capable of binding to only one functionality at a time. Obviously, the exact measurement differs, and is a function of the molecule/type of molecule in question.

Another embodiment of the present invention concerns a microarray presenting chemical molecules, comprising a solid support; a series of wells contained on the solid support; a plurality of linkers attached to each well; a first functionality bound to a portion of the linkers within the plurality of linkers; a second functionality bound to a different portion of the linkers within the plurality of linkers; and a chemical entity bound to at least the first or the second functionality in each well, wherein the majority of wells contain different chemical entities. The functionalities may be spatially separated, as above. The chemical entities may correspond to chemicals present in a chemical library, optionally a random chemical library, or a plurality of chemical libraries. In the case of a random chemical library, the library may contain molecules capable of different types of capture chemistry. In addition, the chemical molecules presented on the microarray may each be bound by at least one of the different functionalities within their respective well; this embodiment also affords the possibility that the chemical molecules presented on the microarray are not all capable of binding to the same functionality in their unbound state.

By “library” in the context of the above chemical libraries, is meant preferably at least 5 chemical entities which differ from each other.

The terms “chemical compound”, “chemical entity”, “small molecule”, “chemical molecule” “small chemical molecule” and “small chemical compound” are used interchangeably herein and are understood to refer to chemical moieties of any particular type which may be synthetically produced or obtained from natural sources and typically have a molecular weight of less than 2000 daltons, more preferably less than 1000 daltons or even less than 600 daltons.

Another embodiment of the present invention provides for a microarray comprising a plurality of functionalities attached to a solid support, wherein the plurality of functionalities is attached to the microarray by a plurality of linkers, and further wherein each different functionality is bound to a different portion of the plurality of linkers The functionalities may be spatially separated, as above. Said microarray may further comprise chemical entities bound to the plurality of functionalities, wherein the majority of wells contain different chemical entities.; in one possible application of this embodiment, the chemical molecules bound to the functionalities are not all capable of being bound by the same functionality in their unbound state. Specifically, any given chemical entity may be bound to one or more or two or more of: the same functionality in different wells; different functionalities in different wells; the same functionality in the same well; different functionalities in the same well; and any combination of these options, all depending on the properties of the chemical entity and the microarray in question. These chemical entities may correspond to chemicals present in a chemical library, all as above.

In a further aspect of the above embodiment, the solid support of the microarray comprises glass and the linkers comprise a silane compound.

The microarrays of the present invention can be prepared using many potential solid supports, such as, inter alia, membranes, FAST slides, and Dendrimer-activated solid supports. Glass or silica has yielded the best results to date (Lam, K S, and Renil, M. (2002). From combinatorial chemistry to chemical microarray. Curr. Opin. Chem. Biol. 6:353-358.); This is because glass has low intrinsic fluorescence properties and can be readily functionalized with a variety of commercially-available reagents useful in coupling small molecules to its surface. In addition, glass has good mechanical, thermal and chemical stability, and is relatively inexpensive.

In a further aspect of the above embodiment, the functionalities of the aforementioned microarray comprise any combination of functionalities selected from the functionality group consisting of, inter alia: alcohol, phenol, aldehyde, ketone, carboxylate, amino group, aryl halide, alkyl halide and sulfhydryl.

In an additional embodiment, the present invention provides a process of preparing a multi-functional microarray comprising attaching a plurality of functionalities to a solid support through a plurality of linkers, wherein the solid support is divided into wells and further wherein at least one functionality of the plurality of functionalities is represented in each well, wherein at least two functionalities of the plurality of functionalities are represented in each well, wherein at least three of the plurality of functionalities are represented in each well, or wherein at least four of the plurality of functionalities are represented in each well, etc. The functionalities may be spatially separated, as described above; this embodiment also provides for a microarray prepared according to these processes.

In another aspect of the present invention, said process may further comprise binding chemical entities to the microarray by reacting them with the plurality of functionalities, wherein only one chemical entity is bound in each well. The chemical entities may correspond to chemicals present in a chemical library, a random chemical library or a plurality of chemical libraries, all as described above. This embodiment also provides for a microarray prepared according to the above processes.

The methods of manufacturing small molecule microarrays as disclosed herein can be performed with already-existing small molecules, with molecules derived from well-established combinatorial synthetic methodologies (on-bead or off) known in the art, with natural product libraries, and with existing compound libraries.

A further embodiment provides for a microarray prepared according to any one of the above processes, wherein the solid support comprises glass and the linkers comprise a silane compound.

A further embodiment provides for a microarray prepared according to any one of the above processes, wherein the functionalities on said microarray comprise any combination of two or more or 3 or more or 4 or more or 5 or more or 6 or more, etc., functionalities selected from the functionality group consisting of, inter alia: alcohol, phenol, aldehyde, ketone, carboxylate, amino group, aryl halide, alkyl halide and sulfhydryl.

The present invention further provides a method of screening a microarray comprising preparing a microarray, preferably a SMA, according to any one of the above processes, and screening said microarray with at least one probe, possibly a polypeptide probe. Said probe could also comprise, inter alia: an oligonucleotide or polynucleotide, which may comprise a promoter sequence or an antisense fragment of a particular gene of interest; a polypeptide which may be a dominant negative peptide or an antibody. Any of these probes may be obtained naturally or synthetically according to methods known in the art. In the case where the probe is a polypeptide, said polypeptide may be known to be the product of a gene involved in a particular disease condition, possibly a mutated gene associated with a disease condition, or the polypeptide itself (or lack thereof) may be known to have an adverse effect in a disease condition.

By the term “probe”, as used herein, is meant any molecule used to interrogate a microarray, in particular a SMA, in order to determine whether a chemical entity having a desired property is bound to the microarray.

In addition, the present invention provides a method of screening a microarray comprising obtaining any one of the microarrays described herein and screening said microarray with at least one probe as described above.

The small molecule microarray of the present invention, as described herein, can be screened with two or more polypeptide molecules simultaneously, provided they do not interact with each other and the detection of one is possible without interference from the other. One possibility is to derivatize the two protein probes with separate fluorophores having sufficient spectral resolution (i.e. Cy3 and Cy5) to permit independent scanning and image processing, as is commonly done with cDNA microarrays. In addition, the small molecule microarrays of the present invention could be reused for screening one or more times following detergent washing and/or enzymatic treatments to completely remove any probe(s) remaining from a prior experiment.

By the term “polypeptide” is meant a molecule composed of amino acids and the term includes peptides, polypeptides, and proteins.

The term “amino acid” refers to a molecule which consists of any one of the 20 naturally occurring amino acids, amino acids which have been chemically modified, or synthetic amino acids. Examples of chemical modification include, inter alia, natural modifications such as post-translational modifications, or modification by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

The term “nucleic acid” refers to any molecule which comprises two or more of the bases guanidine, citosine, timidine, adenine, uracil or inosine, inter alia, or chemical analogs thereof. The term encompasses “oligonucleotides” and “polynucleotides”.

By “disease condition” is meant an alteration in the state of the body or any of its organs, or a disruption in the performance of its vital functions; an individual afflicted with a disease state is unable to perform at least one of the functions he was able to perform before the onset of the disease state, and/or unable to function normally.

An additional aspect of the present invention provides for the use of a chemical molecule identified according to the screening methods described above, for the preparation of a medicament.

Another aspect of the present invention concerns a method of preparing a pharmaceutical composition, comprising identifying a chemical molecule which binds to a polypeptide according to the screening methods described above, and admixing the identified chemical molecule or an analog thereof with a pharmaceutically acceptable carrier.

By “chemical analog” as used herein is meant a molecule derived from the originally identified chemical molecule, that retains the binding activity (to the probe), more particularly that retains the specific activity observed in the parent molecule; chemical analogs may also share structural properties with the parent molecule.

An additional aspect of the present invention concerns screening and testing applications of the multi-functional microarrays disclosed herein, in their capacity to detect the presence of various compounds or entities in a sample. In this case, the microarray comprises a plurality of functionalities attached to a solid support, wherein the plurality of functionalities is attached to the microarray by a plurality of linkers, further wherein only one type of functionality (capable of only one type of capture chemistry) is bound to the plurality of linkers in each well. The sample is typically distributed equally between the wells; a reaction in any of the wells will indicate the presence in the sample of a compound capable of binding and/or chemically reacting with the particular functionality bound in the well wherein the reaction occurred. A non limiting example of this embodiment is that of testing water samples for quality and contaminants; other fluids may be tested for quality and contaminants in a similar fashion.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New York (1989, 1992), and in Ausubel et al., Current Protocols in Molecular Biology, John Wiley and Sons, Baltimore, Md. (1988).

Standard organic chemistry methods known in the art not specifically described herein are generally followed essentially as in Mcmurry, Organic chemistry, Brooks/Cole, Pacific Grove, Calif. (1999 5th ed.); Bruice, Organic chemistry, Prentice Hall Upper Saddle River, N.J. (2001 3rd ed.) and Fessenden et al., Organic chemistry, Brooks/Cole, Pacific Grove, Calif. (1998 6th ed.).

Standard Organic synthesis protocols known in the art not specifically described herein are generally followed essentially as in Organic synthesis: Vol.1-79, editors vary, J. Wiley, New York. (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Example 1

### Preparation of Multi-Functional Microarrays

In general, microarrays can be prepared essentially as described in: Schena et al., Parallel human genome analysis: Microarray-based expression monitoring of 1000 genes. Proc. Natl. Sci. USA (1996) 93, 10614-10619; U.S. Pat. Nos. 6,291,170 and 5,807,522 (see above); U.S. Pat. No. 6,037,186 (Stimpson, inventor). “Parallel production of high density arrays”; PCT publications WO 99/13313 (Genovations Inc [US], applicant) “Method of making high density arrays”; WO 02/05945 (Max-Delbruck-center for molecular medicine [Germany], applicant) “Method for producing microarray chips with nucleic acids, proteins or other test substrates”.

The Steps of Preparation of a Multi-Functional Microarray are Essentially as Follows:

I. Obtaining/Preparing the Solid Surface

Solid surfaces appropriate for microarray preparation have been widely reported in the art (see, for example: Lam, K S, and Renil, M. (2002): From combinatorial chemistry to chemical microarray. Curr. Opin. Chem. Biol. 6:353-358.).

II. Preparing/Obtaining Different Functionalities and Linkers

Linkers of various types can be commercially obtained from various sources. In addition, linkers can be synthesized according to methods known in the art, as described in the references cited above.

There are two possibilities to obtaining the desired functionalities. One is to select commercially available linkers which contain the desired functionality; the other is to chemically modify the selected or synthesized linkers to display the appropriate functionality in an appropriate position.

Synthesis is conducted essentially as described in the above references and particularly, for example, in “Organic synthesis” (see above), or through standard modification and routine optimization of the described therein.

III. Attaching the Linkers to the Solid Surface

The linkers are attached to the solid surface according to chemical procedures known in the art and appropriate for the specific choice of solid surface and linker; appropriate reactions will be easily selected by one of skill in the art, as described in the references cited above.

It will be understood that the preparation steps of the multi-functional microarray of the present invention can also be performed in a different order. For example: the linkers could be attached to the solid surface and the functionalities could then be subsequently attached to the linkers, etc.

The method as described above will be illustrated as follows.

Preparation of a Glass-Based Multi-Functional Microarray With Silane Linkers

1. Selection and Preparation of Glass

There are a number of different types of glass with different physical and chemical properties. Several glass slides are evaluated in order to select the preferred type.

The glass surface is thoroughly cleaned and presents a uniform field of silanol (SiOH), for further derivatization. There are two known methods for the derivatization:

a. Treatment of the glass for 16 h in “piranha solutions,” consisting of 70/30 (v/v) H₂SO₄/H₂O₂, followed by extensive washing in H₂O (Hergenrother, P J, Depew, K M, and Schreiber, S L (2000). Small molecule microarrays: Covalent attachment and screening of alcohol-containing small molecules on glass slides. J. Am. Chem. Soc. 122:7849-7850.).

b. Plasma Etching Techniques.

Fluorescence background, saturation, and impact on dynamic range are all compared in various model systems (for example: model system that consist of a specific protein and a small molecule uniquely recognized by that protein, such as biotin, allopurinol, and pyridoxamine (vitamin B-6) that are uniquely recognized, respectively, by the proteins streptavidin, xanthine oxidase, and aspartate transaminase) on slides from various manufacturers prepared using both procedures.

2. Preparation of Glass Surfaces Presenting Functionalities

The silanol present on the glass surface is reacted with a specific triethoxysilane linker containing an active chemical moiety that subsequently forms a covalent bond with a printed small molecule directly upon contact. The chemical reaction is outlined in the equation below:

Si—OH+(CH₃CH₂O)₃-Si—Z=→Si—O—Si—Z

where (CH₃O)3-Si—Z represents a triethoxysilane with a Z moiety capable of reacting efficiently and irreversibly with a specific chemical functionality should it be present on a small molecule.

There are a wide variety of commercially available silanes suitable to serve as linkers, and specific additional silanes may be synthetically prepared according to methods known in the art.

Silane selection is performed essentially according to the following parameters: specificity and efficiency in reacting with a desired functional group upon contact with a deposited small molecule, minimal elevation of background fluorescence (not emanating from the probe), and compatibility of any such reaction with all subsequent procedures to be performed on the SMA.

2.1. Preparation or Choice of Specific Functional Group “Capture Chemistries” (Functionalities).

The silane linkers selected or created match the chosen functionalities in the small chemical molecules, and are capable of reacting with the small chemical molecules through these functionalities, according to chemistry protocols known in the art.

Amino Group Capture Chemistry

Amino group-based capture of small molecules is performed by reacting the amino groups present on the small molecules with reactive N-succinimidyl esters borne on the silane linkers. (In this case, the “Z” moiety as described above would be defined as the N-hydroxysuccinimide ester.). The silanol contained in the glass will therefore be reacted with a triethoxysilane containing an activated N-hydroxysuccinimide ester.

Sulfhydryl Group Capture Chemistry

Sulfhydryl-based capture of small molecules is performed by reaction with surface-bound maleimido functionalities. The surface silanol is reacted with a triethoxysilane containing a reactive maleimido group (at the end of a linker arm whose length can vary). Another possibility is essentially as described in MacBeath et al. (see above).

Alternatively, sulfhydryl capture is accomplished by reacting the silanol with commercially available 3-mercaptopropyltrimethoxysilane, generating a surface of thiols at the end of a three carbon tether, which is subsequently coupled to the printed small molecules by disulfide bond formation upon simple oxidation.

Another possibility is to present a surface of halo-amides or pyridyldisulfides. A displacement reaction of the pyridylthiol with HS—R is performed, and the candidate is thus bound covalently to the glass. (Sulfhydryl groups, especially the thiolate anion, are very active nucleophiles).

Alcohol Group Capture Chemistry

Alcohol group-based capture of small molecules is performed essentially as described in Hegenrother et al (see above).

Aryl Halide Capture Chemistry

Aryl halide group-based capture of small molecules is performed by a Suzuki-based coupling procedure based on the reaction of aryl halides with surface-bound phenyl boronate. The silanol is converted to the appropriate phenyl boronate derivitive (in the presence of a palladium catalyst), which then reacts with the appropriate small molecules.

2.2. Preparation of a Multi-Functional Microarray Comprising the Above Described Functionalities

In order to prepare a multi-functional microarray, an excess of different silanes displaying or containing any combination of the above functionalities is mixed together and bound to the glass surface; as the functional group does not influence the binding of the silane to the glass (as is evident from the chemical equation above), the majority of wells will contain all the selected functionalities in equal proportions. The binding is conducted as described above.

2.3. Optimizing Surface Reactive Silanes Within a Matrix of an Inert Silane to Achieve Spatial Separation

The reactive silane that binds to the surface silanol in the glass is added to the glass at various concentrations in conjunction with an inert silane that adjusts the concentration of reactive groups on the surface accordingly, and thus spatial separation is achieved. (This separation can be important for subsequent screening of the array: a surface too densely packed with small molecule ligands may not be optimal for probe binding; one with too little may not produce sufficient signal during detection).

By “in conjunction with” is meant prior to, simultaneously or subsequent to.

By “inert” is meant lacking any functionality, and thus incapable of chemically reacting with another moiety.

An example of an inert silane is, inter alia, n-propyltriethoxysilane.

### Example 2

### Preparation of Chemical Libraries/Chemical Compounds

Chemical compounds can be prepared essentially according to protocols that appear in the above cited references, particularly in the series: “Organic synthesis” (see above), with reagents obtained for example from Sigma-Aldrich. New or derivatized chemical compounds are typically obtained by optimizing the procedure for a known chemical compound.

In addition, entire chemical libraries can be purchased from, inter alia: Chembridge (San Diego, Calif. USA). Asinex (Moscow, Russia), and Chemdiv (San Diego, Calif., USA).

### Example 3

### Preparation of Chemical/Small Molecule Microarrays

Small molecule microarrays can be prepared essentially as described in Gregory et al.: Journal of the American Chemical Society; 2001; 123(2); 361-362; Hergenrother et al., Journal of the American Chemical Society; 2000; 122(32); 7849-7850; MacBeath et al., Journal of the American Chemical Society; 1999;121(34); 7967-7968; Kuruvilla et al (Nature 416 (2002), 653-657.

Binding of Chemical Molecules to a Multi-Functional Microarray

The chemical molecules synthesized according to the above can be bound to the multi-functional microarray of the present invention essentially according to standard chemistry protocols.

Specifically, each chemical molecule will bind to the functionality or functionalities that it is capable of binding to out of the functionalities presented on the microarray; through routine optimization of protocols known in the art, it is possible to carry out several chemical binding reactions simultaneously.

Another approach to the preparation of small molecule microarrays, also considered to be a part of the instant invention, is to first bind different types of functionality bearing linkers to small molecules or a small molecule library, and subsequently printing or attaching the linkers onto an appropriate solid surface. In this case the linkers serve as tethers to the surface.

Blocking and Capping of Unreacted Groups

After attaching the small molecules to the microarray, a blocking step is employed to the surface, in order to prevent the possibility of the probe reacting with unbound functionalities (For example, polypeptide probes may react via covalent couplings with several functionalities, such as, inter alia: alcohols (in serines and threonines), aminos (in lysines), and sulfhydryls (in cysteines)).

For each functional group, an appropriate blocking agent is chosen. Examples of blocking agents that may be employed are: β-mercaptoethanol, that effectively reacts with any maleimido groups; methyl or ethylamine are employed against any unreacted N-succinymidyl esters; methyl or ethyl alcohol or even ethylene glycol are used to react with any alcohol-reactive functionalities.

### Example 4

### Preparation of Polypeptides

Polypeptides may be produced via several methods, for example:

1) Synthetically;

Synthetic polypeptides can be made using a commercially available machine, using the known sequence of the desired polypeptide.

2) Recombinant Methods:

A preferred method of making polypeptides is to clone a polynucleotide comprising the cDNA of the gene of the desired polypeptide into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965;23:1165-74; Appl Microbiol. 1967 July;15(4):851-6; Can J Biochem. 1968 May;46(5):441-4; Biochemistry 1968 July;7(7):2574-80; Arch Biochem Biophys. 1968 Sep. 10;126(3):746-72; Biochem Biophys Res Commun. 1970 Feb. 20;38(4):825-30).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification From Natural Sources:

Desired polypeptides can be purified from natural sources (such as tissues) using many methods known to one of ordinary skill in the art, such as for example: immuno-precipitation, or matrix-bound affinity chromatography with any molecule known to bind the desired polypeptide. Protein purification is practiced as known in the art as described in, for example. Marshak et al., “Strategies for Protein Purification and Characterization. A laboratory course manual.” CSHL Press (1996).

### Example 5

### Screening of Microarrays

Small molecule microarray analysis is performed essentially as described in the references incorporated herein. See for example: Gregory et al.: Journal of the American Chemical Society; 2001; 123(2); 361-362; Hergenrother et al., Journal of the American Chemical Society; 2000; 122(32); 7849-7850; MacBeath et al., Journal of the American Chemical Society; 1999;121(34); 7967-7968; Kuruvilla et al (Nature 416 (2002), 653-657.

Probes

General methods and conditions of screening microarrays are known in the art. As described herein, many types of molecules can serve as a screening probe; the conditions of the binding reaction between the probe and the microarray should therefore be tailored to the probe being used.

One preferred probe is a polypeptide probe. In addition to the standard methods known in the art for screening a SMA with a polypeptide probe (some of which are referenced above), it is important to recognize that most proteins have some capacity to adsorb onto almost any surface, due to localized hydrophobic, hydrophilic, and ionic domains on the surface of the polypeptide. This ability to adsorb non-specifically can be minimized by using sufficient concentrations (1-10 mg/ml) of an inert, non-detectable protein such as bovine serum albumin (BSA) or casein in wash buffers and during subsequent hybridization to labeled polypeptide probes.

Detection Systems

In order to effectively screen the small molecule microarrays of the present invention, a system to specifically detect binding of the probe to a small molecule is necessary. One detection system employed with the present invention is the measurement of fluorescence emanating from dye-conjugated probes, in several possible variations. For each probe being used in the screening methods of the present invention, it is necessary to conjugate the dye in a manner that does not interfere with small molecule binding. Several examples are: 1. Performing the dye labeling in the presence of an excess of the small molecule, provided that it doesn't have amino groups. 2. Biotinylating the probe using non-amino conjugation chemistry, following with fluorescent-labelled streptavidin. 3. In the case of a polypeptide probe only, performing the dye labeling on an antibody complementary to the polypeptide probe, and measuring fluorescence of the immunological complex.

The dyes Cy3, Cy5, and Alexa, inter alia, are conjugated to the probe as known in the art. Reagents, protocols, and complete kits needed to conjugate these dyes to polypeptides, including antibodies, are available from several manufacturers including BioRad, Molecular Probes, and Amersham Pharmacia Biotech.

In the case of a polypeptide probe, an additional option for fluorescent detection is constructing fusion proteins of the desired probe with green fluorescent protein (GFP), as well known in the art.

The scanning of microarrays for fluorescence emission is performed with an Axon Instruments Model 4000 fluorescent scanner; the data is processed using GenePix software.

Additional detection systems include, inter alia: radioactively tagging the probe with an appropriate isotope according to methods known in the art; for polypeptide probes: alkaline phosphatase assay, horse-radish peroxidase assay, and immuno-detection with specific antibodies.

Possible Re-Cycling

In order to re-cycle the SMA for re-probing, the previous probe must be stripped from the microarray. This can be accomplished according to methods known in the art, depending on the type of probe that needs to be stripped.

Validation

Validation of the binding between the chemical molecule on the microarray and the probe can be done by methods known in the art.

The small molecule that bound to the probe can be identified according to its location on the microarray, as every well contains only one type of molecule. Following the identification, the small molecule can be prepared (or extracted from the original library) and a validation binding assay can be conducted in vitro between the probe and the small molecule, under the appropriate conditions and with the same detection system as in the case of the entire microarray, or with other detection systems known in the art.

In addition, the effect of the identified small molecule on the biological activity of the probe can be examined.

In case of a polypeptide probe, a known activity of the polypeptide can be measured before and after/with or without binding to the small molecule. Detectable biological activities include enzymatic activities and binding (to molecules the polypeptide is known to bind in-vivo or in-vitro).

In case of a polynucleotide probe, the effect of the small molecule on the transcription or protein expression of the endogenous gene can be examined in-vivo in an appropriate cell culture system.

1. A multi-functional microarray comprising the following elements:
a) a solid support;
b)) a series of wells contained on the solid support;
c) a plurality of linkers attached to each well;
d) a first functionality bound to a portion of the linkers within the plurality of linkers; and
e) a second different functionality bound to a different portion of the linkers within the plurality of linkers.

2. The microarray of claim 1 wherein the functionalities are spatially separated.

3. The microarray of claim 2 wherein a portion of the plurality of linkers have no bound functionality, thereby achieving spatial separation between the functionalities.

4. A microarray presenting chemical molecules comprising the following elements:
a) a solid support;
b) a series of wells contained on the solid support;
c) a plurality of linkers attached to each well;
d) a first functionality bound to a portion of the linkers within the plurality of linkers;
e) a second different functionality bound to a different portion of the linkers within the plurality of linkers; and
f) a chemical entity bound to at least the first or the second functionality in each well, wherein the majority of wells contain different chemical entities.

5. The microarray of claim 4 wherein the functionalities are spatially separated.

6. The microarray of claim 5 wherein a portion of the plurality of linkers have no bound functionality, thereby achieving spatial separation between the functionalities.

7. The microarray of claim 4 wherein the chemical entities bound to the wells correspond to chemicals present in a chemical library.

8. The microarray of claim 7 wherein the chemical library is a random chemical library.

9. A microarray comprising a plurality of functionalities attached to a solid support, wherein the plurality of functionalities is attached to the solid support by a plurality of linkers and further wherein each different functionality is bound to a different portion of the plurality of linkers.

10. The microarray of claim 9 wherein the functionalities are spatially separated.

11. The microarray of claim 10 wherein a portion of the plurality of linkers have no bound functionality, thereby achieving spatial separation between the functionalities.

12. The microarray of claim 9 further comprising chemical entities bound to the plurality of functionalities, wherein the majority of wells contain different chemical entities.

13. The microarray of claim 12 wherein the chemical entities correspond to chemicals present in a chemical library.

14. The microarray of claim 13 wherein the chemical library is a random chemical library.

15. A microarray according to any one of claims 1, 4, 9 or 12 wherein the solid support comprises glass and the linkers comprise silane compounds.

16. A microarray according to any one of claims 1, 4, 9 or 12 wherein the functionalities are any combination of alcohol, phenol, aldehyde, ketone, carboxylate, amino group, aryl halide, alkyl halide or sulfhydryl.

17. A process of preparing a multi-functional microarray comprising attaching a plurality of functionalities to a solid support through a plurality of linkers, wherein the solid support is divided into wells and further wherein at least one functionality of the plurality of functionalities is represented in each well.

18. A microarray prepared by the process of claim 17.

19. The process of claim 17 further comprising spatially separating the functionalities.

20. The process of claim 19 wherein a portion of the plurality of linkers have no bound functionality, thereby achieving spatial separation between the functionalities.

21. The process of claim 17, further comprising binding chemical entities to the microarray by reacting them with the plurality of functionalities, wherein only one chemical entity is bound in each well.

22. The process of claim 21 wherein the chemical entities correspond to chemicals present in a chemical library.

23. The process of claim 22 wherein the chemical library is a random chemical library.

24. A microarray prepared by the process of claim 21.

25. A method of screening a microarray comprising preparing a microarray according to the process of claim 21 and screening said microarray with at least one probe.

26. The method of claim 26 wherein the probe is a polypeptide.

27. A method of screening a microarray comprising screening the microarray of any one of claims 4 or 12 or 24 with at least one probe.
